(19) United States
(12) Patent Application Publication
(10) Pub. No.: US 2003/0027221 A1
(43) Pub. Date: Feb. 6, 2003

(54) High-throughput screening assays by encapsulation

(76) Inventors: Melissa E. Scott, Stanford, CA (US); Haim Kedar, Palo Alto, CA (US); Andrew J. Kelly, Palo Alto, CA (US); David E. Huber, Mountain View, CA (US); Richard B. Mortensen, Menlo Park, CA (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 09/828,072

(22) Filed: Apr. 6, 2001

(51) Int. Cl. C12Q 1/68; G01N 33/567; G06F 19/00; G01N 33/48; G01N 33/50

(52) U.S. Cl. 435/7.21; 435/6; 702/19; 702/20

(57) ABSTRACT

Candidates for biological activity are screened in capsules, each capsule containing a candidate, a target (which may be a molecular species or a biological cell or multiple copies of such a species or cell), and an intelligent substance. The target is one with which a successful candidate will interact to evoke a particular response in the target, and the intelligent substance is one that undergoes a transformation as a result of the response, the transformation being detectable by observation of the capsule itself. The candidate and the target are isolated from each other in the capsule until a designated point in time thereby enabling the operator to control the time interval between exposure of the target to the candidate and the observation of the capsule to determine whether or not a successful interaction has occurred and the transformation has taken place.

### BACKGROUND OF THE INVENTION

1. Field of the Invention

This invention resides in the field of drug screening, and in particular to methods for testing candidate species, including small molecules, macromolecules, biological species, viruses, and bacteria and other organisms, as well as combinations of distinct molecules, macromolecules or other individual species, for their ability to evoke a particular response in a target molecule or biological material.

2. Description of the Prior Art

The state of the art in the biotechnology and pharmaceuticals industries is continually being advanced as research into diseases and disease conditions provides new understandings of the etiologies, cellular origins, biological mechanisms, and pathways that give rise to, control, or prevent these conditions. These new understandings prompt the industry to seek and develop new drugs and biologically active substances in general as candidates for the prevention, control and treatment of disease. One of the challenges that these research efforts face is to screen candidate substances for the desired activity, and to do so in a reliable and yet economically efficient manner. The challenge is a particularly great when the number of candidates to be screened is very large.

Early efforts at seeking drug candidates were directed to substances obtained from natural sources such as bacteria, fungi, invertebrates and plants. Screening, although done on a random basis, achieved considerable success—over a hundred biologically active materials which are currently being used as antibiotics, agricultural chemicals, and anti-cancer agents have been developed in this manner. With the relatively recent advent of combinatorial chemistry, however, synthetic candidates can now be produced in much larger numbers, creating large libraries of synthetic chemical species and of genetically engineered microorganisms as candidate pools, greatly increasing the number and variety of candidates available for screening.

In the most common screening procedures, bioassays are performed on each candidate, exposing the candidate to whole cells or organisms to determine whether the candidate elicits the cellular activity or response that is believed to be causative of or preventive of the condition being investigated. The success of these methods depend on such factors as the reliability of the bioassay, the number and diversity of the candidates, the number of assays that can be performed in parallel, and the ability to distinguish between meaningful responses on the one hand and false positives or background noise on the other.

For the screening of naturally occurring compounds, the candidate pools have typically contained as many as hundreds of thousands of candidates. The use of these compounds as candidates is limited of course by difficulties in finding the compounds, isolating them, determining their structures, reproducing them, and supplying them for testing. This plus the limited number of natural sources has resulted in a diminution of the number of naturally occurring compounds that are available for screening. Combinatorial chemistry has overcome this problem by removing many of these limitations and thereby increasing the number and variety of candidate compounds. Thus, there is a continuing need for screening procedures that can accommodate large libraries of candidate compounds.

Of potential relevance to this invention are uses and disclosures of methods for immobilizing biological cells. The immobilization of biological cells has been anintegral part of various procedures involving investigations of biotransformations, transplantations, clinical microbiology, toxicology, food chemistry, and environmental sciences. Investigations involving immobilized cells has resulted for example in the discovery and development of antibiotics such as certain penicillins, bacitracin, erythromycin, and oxytetracycline. Disclosures are found in Deo et al., Biotechnol. Bioeng. 26: 285-295 (1984); Flanagan et al., Biotechnol. Bioeng. 36: 608-616 (1990); Morikawa et al., Biotechnol. Bioeng. 22: 1015-1023 (1980); Bandyopadhyay et al., Biotechnology Letters 15: 1003-1006 (1993); Weaver, U.S. Pat. No. 4,309,219, Aug. 6, 1983; Weaver, U.S. Pat. No. 4,401,755, Aug. 30, 1983; Weaver, U.S. Pat. No. 4,916,060, Apr. 10, 1990; Weaver et al., U.S. Pat. No. 4,959,301, Sep. 25, 1990; Weaver et al., U.S. Pat. No. 5,055,390, Oct. 8, 1991; Cochrum et al., U.S. Pat. No. 5,578,314, Nov. 6, 1996; Chromaxome Corporation, International Patent Application No. WO 98/41869, published Sep. 24, 1998; and Diversa Corporation, International Patent Application No. WO 98/58085, published Dec. 23, 1998.

### SUMMARY OF THE INVENTION

It has now been discovered that candidate species of various kinds can be screened for their ability to interact with a target and produce a useful result, and that the screening can be performed in a highly controlled manner by encapsulating the candidate species, the target (or multiple targets), and an intelligent substance in a common capsule in a manner that will keep the candidate and target isolated from each other until the capsule is subjected to an externally imposed condition that permits or causes the candidate to contact the target. Intelligent substances are substances that respond to a stimulus in a detectable manner, and in the present invention the stimulus is a change in the environment of the substance within the capsule, or a change in a particular aspect of the environment, the change being caused by a response that the target displays when the candidate is one that has the characteristics being sought. In certain implementations of the invention, the external condition is imposed at a distinct and selected point in time, enabling one to control the time at which the stimulus is applied to the intelligent substance and the time and manner in which the transformation, if any, is detected. This improves the accuracy of the determination of whether a transformation has taken place and facilitates the means by which capsules whose intelligent substances have been transformed are distinguished from those whose intelligent substances have not been transformed, and thus the means of determining whether any particular candidate is biologically active in terms of the particular response of the target. Aside from timing considerations, the encapsulation provides ease of use and handling, effectively isolating the assay medium from its surroundings and from the assay media of other candidates. Encapsulation also eliminates the need for separating solid and liquid phases and other such manipulations that are often required in heterogeneous assays.

By combining encapsulation with the ability to manipulate the barrier that isolates the candidate species from the target, the present invention permits one to design systems with a wide range of detection techniques and a high degree of accuracy in distinguishing candidates that demonstrate the desired interaction from those that do not. The invention also permits a large number of capsules to be formed and treated simultaneously under uniform conditions before the transformation occurs. The encapsulation also lends itself to miniaturization, with capsules on the millimeter, micron or nanometer scales. Consequently, a large number of candidates can be assayed simultaneously. The use of an intelligent substance permits the detection of active candidates and the differentiation between those that are active and those that are not without isolating or separating individual capsules from each other.

These and other features, embodiments, objects and advantages of the invention will be better understood from the description that follows.

### DETAILED DESCRIPTION OF THE INVENTION AND SPECIFIC EMBODIMENTS

Candidates that can be screened in accordance with this invention include any species that can be encapsulated and that are potential candidates for interaction with a target in a manner that provides a useful result, either in terms of diagnosis or therapy or in generating information that leads to a greater understanding of biological, physiological, or chemical function. The candidates may thus be small molecules, macromolecules, peptides, oligonucleotides, polynucleotides, oligomers, polymers, analogs of peptides, analogs of oligo- and polynucleotides, liposomes, and other chemical compounds, as well as biological species such as antibodies, enzymes, viruses, bacteria, fungi, and biological cells, cell fragments and cell products in general. Candidate species may also be combinations of small molecules, combinations of peptides, combinations of small molecules and peptides, combinations of oligo- or polynucleotides, or combinations of other types of species. One area in which the invention is particularly useful is in the screening of synthetic chemical compounds, notably those produced by combinatorial chemistry, such as peptides, oligonucleotides and polynucleotides and analogs thereof, and other oligomers and polymers. Other areas will be readily apparent from the detailed descriptions given below.

The target may be any molecular or biological entity that produces a beneficial or otherwise useful result upon interaction with an appropriate candidate. Molecular entities include small molecules and macromolecules, while biological entities include cells, tissue, cell surface regions, cell components, viruses, bacteria, and the like. The target may thus for example be a protein, a sugar, a polysaccharide, a nucleic acid, a lipid, a cell surface receptor, an intracellular receptor, an enzyme, a transcription factor, or a kinase. Examples of intracellular receptors are estrogen receptors, glucocorticoid receptors, androgen receptors, progesterone receptors, and mineralocorticoid receptors. The capsule may contain a single copy of the target or multiple copies such as individual disconnected cells, fused cells or continuous cell masses such as tissue. The target is selected on the basis of its ability to undergo, exhibit or demonstrate the particular response that is sought in successful candidates, or an analogous response that is representative of the desired response.

The response evoked by the successful candidate and the ensuing change in environment that causes a transformation in the intelligent substance in accordance with this invention may vary widely. Depending on the target, the response may be one that causes a change in the electric field surrounding the target, a change in the pH of the target and its surroundings (by releasing or secreting protons, for example), a change in temperature (such as a lowering of the temperature of a target cell and its surroundings due to consumption of energy by the cell, or a temperature rise due to metabolism occurring within the cell). When the target is a biological cell, the response may be the secretion of an agent from the cell, the agent being either a product of cell metabolism or a species that is cleaved within the cell and released, permitting the species to permeate the cell membrane. Biologically active candidate species will be those that either penetrate the cell membrane and once inside the cell elicit one of these responses, or that cause the cell to elicit one of these responses by binding to a receptor on the exterior of the cell membrane or otherwise interacting with the exterior of the membrane. For targets that are molecules rather than cells, the response may be the release of an agent due to cleavage of the target if the candidate is an enzyme or other cleaving reagent and the target is a substrate for the enzyme or reagent.

The term “intelligent substance” is used herein to denote any substance that responds to an interaction between candidate and target in a detectable manner, and a “successful” candidate is one which interacts with the target in such as manner as to cause the intelligent substance to respond. Examples of intelligent substances are polymers, hydrogels, monomers, solutions of monomers, and colloidal and other suspensions. The manner in which the intelligent substance responds to the interaction can be any of a wide variety of transformations, signal emissions, or reactions in general. Examples are changes in size due to swelling or shrinkage, changes in density, changes in crystal structure, changes in magnetic properties, changes in optical properties such as opacity, refractive index, polarization, or reflectivity, the release or a change in the absorptivity of a detectable agent, or the release or a change in the absorptivity of a detectable signal (such as an electromagnetic signal, radioactivity, fluorescence, light, microwaves, radiofrequency (RF) waves, or ultrasound). The response may for example be the polymerization of a monomer, or an increase in crosslinking of an otherwise uncrosslinked polymer or one that is crosslinked only to a limited extent, or it may be the cleavage of crosslinking sites in an otherwise highly crosslinked polymer. Since crosslinking of a polymer tends to cause a size reduction in the polymer, the removal of (or reduction in) crosslinking in many embodiments of this invention will result in an increase in size of the polymer. With cellular targets, the response may be an increase in cellular metabolism which may cause a shift in intracellular pH. The appropriate intelligent substance may then be one that is pH sensitive. One example of such a substance is hydrolyzed crosslinked polyacrylamide, a weakly ionic polymer that swells when exposed to high pH and diminishes in size at low pH, with a sensitivity of approximately 0.5 pH unit. This polymer is capable of absorbing twenty times its weight in water at pH 7.0, for example, and of releasing 85% of the absorbed water at pH 5.0. The polymer reaches equilibrium in only a few minutes, and the changes are reversible and occur readily upon raising or lowering of the pH of the surrounding environment. Another response with a cellular target may be the activation of a calcium channel or channels for other ions resulting in the release of intracellular reserves of these ions. The change in environment is then a flux in the concentration of the ion, and an example of an appropriate intelligent substance is a polymer that is sensitive to this flux by expanding or diminishing in size. Polymers that exhibit this type of behavior are known to those skilled in polymer chemistry.

The transformation in the intelligent substance is detectable by detecting a corresponding indication in the capsule. The indication may be the same as the transformation in the intelligent substance for example, a change in size of a polymer as by shrinkage or expansion may be transmitted to the capsule as a whole by a corresponding shrinkage or expansion of the capsule, or an emission or absorption of an agent or an emission by the substance may be detected as an emission or absorption of the capsule as a whole. Another example is a change in density, which may also result from a crosslinking change, or by absorption or release of a component from the substance and hence the composition of the capsule as a whole. A third example is the emission of fluorescent energy upon excitation, or of other types of detectable signals. A fourth example is a change in an optical property of the polymer and hence the capsule. The change may for example be a change from a transparent state which transmits incident light to a translucent or opaque state that absorbs incident light, or a change in refractive index which causes in change in the angle of deflection of incident light.

In some of the preferred embodiments of this invention, the intelligent substance is a polymer and the transformation in the polymer is a change in the density or physical size of the polymer due to an increase or reduction in the degree of crosslinking within the polymer. In certain preferred embodiments, the response of the target (whether the target is a cell or other biological material or a molecule) is the release of an agent that disrupts the crosslinking of the polymer. For example, the polymer may be formulated to include both antigens and antibodies within its molecular framework, and the crosslinking may be achieved by an antigen-antibody interaction. When the response is the release of free antigen or antibody, one possible result may be competition between the released member and its counterpart in the polymer structure, the competition causing displacement of the counterpart in the binding interaction, thereby replacing the polymer-bound binding member with free binding member and disrupting the crosslinkage. As another example, the polymer may be crosslinked by a peptide or nucleotide that is cleavable by an enzyme, and the cellular response may be a release of the enzyme. A linking group containing a β-galactoside can for example be cleaved by a β-galactosidase. If the polymer is crosslinked with β-galactoside linkage, therefore, and a successful candidate-target interaction causes release of a β-galactosidase, this release will result in the cleavage of the crosslinker which will in turn cause expansion of the polymer.

Another possible transformation that may occur in accordance with this invention is the emission of a fluorescent signal from the intelligent substance. This may occur for example when the intelligent substance is a polymer or other molecule is conjugated to a substance that becomes fluorescent when liberated. A candidate-target interaction that can be detected in this manner is one that results in the release of a cleaving agent that cleaves the fluorophore from the intelligent substance. The change in environment is then the new presence of the cleaving agent in the medium surrounding the target. The fluorophore may for example be a 5-alkanoylaminofluorescein di-β-galactopyranoside, such as C8-FDG or C12-FDG, and a cleaving agent that will release either of these is β-galactosidase. As another example, the intelligent substance may contain a pair of fluorescent groups spatially arranged to form a fluorescence resonance energy transfer (FRET) pair, i.e., one in which the fluorescent emission of one member of the pair (the donor) is absorbed by the other member of the pair (the acceptor) when the two are fixed at a particular distance from each other or in a particular orientation relative to each other, the result being either a total absorption of the fluorescence or an emission from the acceptor of a fluorescent emission at a different wavelength. A successful candidate-target interaction may then be one that causes a cleavage of the intelligent substance, or in the case of a polymeric substance, a disruption in the crosslinking of the polymer (by any of the means described above), leading to an alteration in the spatial distance between the donor and acceptor or in their relative orientation. When such an alteration occurs, the resonance between the two fluorescent groups is destroyed and the donor fluorescence is no longer absorbed and therefore detectable. Examples of FRET donor/acceptor pairs are fluorescein/tetramethylrhodamine, IAEDANS/fluorescein, EDANS/DABCYL, fluorescein/fluorescein, BODIPY FL/BODIPY FL, and fluorescein/QSY-7. Other FRET donor/acceptor pairs are known in the art.

The capsule can be formed in various ways. The capsule components may for example be enclosed in a shell in which the components, including the candidate species, the target, and the intelligent substance, are mobile. Alternatively, the capsule may be formed by entrapping or embedding either the target or the candidate species or both in the intelligent substance in an immobile manner, as for example in the interstices of a polymer lattice. With the target and/or candidate species thus entrapped or embedded, the polymer matrix may itself form the capsule, optionally surrounded by an encasing shell.

The separation of the candidate species from the target prior to the point at which candidate and target are to be placed in contact can be achieved by a physical barrier between the target and the candidate species that can be removed at will by external means without disrupting the capsule. Alternatively, the components can be kept out of contact by a spatial separation maintained by immobilization of one or both components in different parts of the capsule and yet capable of being released by external means for migration through the capsule. The intelligent substance can form a matrix in which either the target, the candidate species, or both are suspended, or it can form a layer or outer shell adjacent to or surrounding a second matrix in which the target is suspended, or a layer inside the outer shell. For example, the candidate species may be retained in an inner capsule which does not contain the target but is itself retained in an outer capsule which retains the target and the intelligent substance, with the walls of the inner capsule serving as the barrier that is capable of being ruptured or made porous by external means. The space between the inner and outer capsules may be occupied by a lattice or porous network of the intelligent substance in whose interstices the target is lodged. With rupture of the inner capsule walls, the candidate species will pass through the walls and migrate through the interstices or pores of the intelligent substance to contact the target. As another example, particularly for a polymeric intelligent substance, the candidate species may be bonded to a solid support, preferably a bead which, although still within the capsule, resides in a peripheral region of the capsule outside the region occupied by the polymer lattice and is too large to penetrate the polymer lattice, the bond between the species and the bead being cleavable by external means to release the species for diffusion through the polymer lattice. As a variation, the polymer lattice may occupy the peripheral region of the capsule surrounding the bead with the cells occupying a secondary internal capsule. The wall of the secondary capsule is permeable to the candidate species and the target response when evoked penetrates the wall to the polymer network in the peripheral region. As a further example, for embodiments of the invention in which the intelligent substance is a hydrogel that swells or contracts in response to a stimulus, the candidate species may be sequested in the hydrogel when the hydrogel is in a swollen state. The target response may then be a change in pH, temperature, light or other electromagnetic energy, or any of the other stimuli that are known to cause a contraction in certain hydrogels, and the contraction will cause the hydrogel to expel the candidate, thereby permitting the candidate to migrate toward and contact the target. Hydrogels that respond to any of a variety of external stimuli are known in the art. Other configurations and mechanisms will be readily apparent to those skilled in the art.

When beads as described in the preceding paragraph are used, the size of the bead may vary. Preferred beads are those whose longest linear dimension (i.e., the diameter, for spherical beads, and for elliptical beads, the longest axis ) is from about 1 nm to about 1 mm in length. A more preferred size range is from about 0.5 μm to about 500 μm. Beads in the nanometer or micron range are referred to herein as “microbeads.”

When the temporary separation of candidate from target is achieved by a physical barrier, the barrier may be either a partition in the capsule structure or an internal enclosure surrounding one of the components to the exclusion of the other. The barrier can be constructed for rupture by any of a variety of methods. Mechanical rupture of the barrier may be achieved by impact, such as for example by the vibration of pellets or beads within the capsule, or other means of exerting force on the barrier from within the capsule. The barrier may for example be an inner shell with magnetic beads placed inside, and rupture of the shell can be achieved by oscillating the beads under the influence of an alternating magnetic field, causing the beads to forcefully strike the shell from the inside or to align and cause an elongation and subsequent rupture of the shell. Alternatively, the inner shell may contain a particle or member such as a microballoon or pulsatile object that expands or contracts under external influences to a degree sufficient to cause cracks or fissures in the shell that will permit penetration of the shell by the candidate species, or even to burst the shell entirely.

As a further alternative, the physical barrier can be one that is normally nonporous but capable of being made porous by an external influence. This can be achieved, for example, by forming the barrier from a mixture of polymers or a mixture of a polymer and a non-polymeric additive, whereby one of the polymers in the mixture or the additive will either contract or dissolve upon external influence to leave interstices or pores in the remaining polymer and hence in the barrier structure. Other means of rupturing the barrier or making it porous include ultrasound, electroporation (i.e., the use of a barrier that becomes porous upon exposure to an electric field), bioporation (the decomposition of the barrier wall by enzymes or bacteria), dissolving of the barrier wall by a selective solvent, or ablation of the barrier by irradiation with light energy, such as ultraviolet light energy or energy within a narrow wavelength range. Selective disruption of the barrier relative to other materials contained within the capsule can be achieved for example by selecting a barrier composition that absorbs radiation within a limited wavelength range and matching the wavelength range to the barrier composition. One example of a method for disrupting the barrier is disclosed in International Patent Application Publication No. WO 99/59556, entitled “Externally triggered microcapsules,” NASA/Johnson Space Center, applicant; international publication date 14 Nov. 1999 (application no. PCT/US99/10656).

The external influence that causes disruption of the barrier or the conversion of the barrier from a nonporous to a porous material may be any condition, electromagnetic irradiation, the imposition of a magnetic or electric field, a change in solvent, temperature, or pH, the exposure to a chemical reagent or biological reagent such as an enzyme, antibody, inhibitor, antagonist or the like, or any such influence that causes a disruption or porosity increase in the barrier. The choice will depend on the barrier composition and is not critical to the basic concepts of this invention.

Once the transformation in the intelligent substance has occurred, the differentiation between candidate species that effected the transformation and those that did not is achieved by the comparing the capsules for indications of which ones contain transformed substance. This is achieved by any of various means depending on the type of transformation. All of the changes can be detected by conventional means that will be readily apparent to those skilled in the art, the appropriate means in each case being evident from the changes themselves. Fluorescent emissions for example can be detected by conventional fluorescence emissions detectors. Changes in density can be detected in a variety of ways, including buoyancy differentials, settling, and centrifugation. Changes in size, shape or both can be detected by light scattering differences such as those that can be detected by flow cytometry.

In preferred embodiments of this invention, the capsule is microscopic in size, with its longest linear dimension ranging from about 10 nm to about 2 mm, most preferably from about 5 μm to about 500 μm.

In certain embodiments of this invention, the candidate species are multi-unit constructs each consisting of a sequence of linked units, the units differing in structure and the constructs potentially differing in the units included, in the sequence of the units, or both. Constructs of this type can be synthesized in libraries (large numbers of different constructs produced simultaneously) by combinatorial techniques that are well known among those skilled in combinatorial chemistry, a rapidly developing field with much published literature. Examples of constructs that can be synthesized in this manner are linear, cyclic, and branched oligomers and polymers of nucleic acids, polysaccharides, phospholipids, peptides (including alpha-, beta-, or omega-amino acids or combinations thereof), and polymers with other types of linkages, such as polyurethanes, polyesters, polycarbonates, polyureas, polyamides, polyethyleneimines, polyarylene sulfides, polysiloxanes, polyimides, and polyacetates.

The typical library will contain a multitude of microbeads, each bead having formed thereon a different construct, with a single construct per bead. A typical method of forming such a library involves the steps of (a) apportioning the microbeads randomly among different reaction vessels; (b) adding monomeric units to each vessel to bond to the beads, using a single monomeric unit for each vessel; (c) recovering the microbeads (to each of which a single monomeric unit has been bound) from the reaction vessels and pooling the recovered microbeads; (d) reapportioning the microbeads among different reaction vessels, (e) adding monomeric units to each vessel, again in a random manner, for bonding to the first monomeric units that are already bonded to the microbeads; and repeating the pooling, reapportioning, and bonding steps a sufficient number of times to achieve sequences of the desired length on each microbead. The result will be a different sequence on each microbead, and the number of units in each sequence may vary widely, depending on the type of library desired. In most cases, the number of units in each sequence will range from 3 to 300. The bonding reaction and the type of linkage between units on each microbead in a single cycle will often be the same, with the only difference from one microbead to the next-being the units themselves or the order in which they are linked together. The number of microbeads in each reaction vessel may vary as well, from as little as one to a large number. In general, the various reaction vessels during each cycle of the process will contain substantially equal numbers of microbeads. Conventional reaction conditions and procedures well known to those skilled in synthetic chemistry will be used, the particular procedure and set of conditions for any particular type of polymer being selected for the linkage used in that polymer. Thus, for polyurethanes, typical conventional polyurethane linking techniques will be used for each monomeric unit; for polyesters, typical conventional polyester linking techniques will be used; for polyimides, typical conventional polyimide linking techniques will be used; for peptides, typical peptide chemistries will be used; for nucleic acids, typical oligonucleotide forming chemistries will be used; and so on for each of the various polymers.

The various constructs formed in this manner can be identified by conventional analytical chemistry techniques. In general, the successful candidate(s) will be analyzed to determine its structure. A particularly convenient means of identification, however, is the use of identifier tags associated with each microbead. A wide variety of encoding technologies known in the art of combinatorial chemistry can be used. One such technology is encoding with DNA, useful for example in peptide libraries by binding a unique, single strand of DNA to each different peptide, with specific sequences of DNA assigned to each building block or monomer. A description of DNA encoding appears in Brenner, S., et al., “Encoded combinatorial chemistry,” Proc. Natl. Acad. Sci. USA 89: 5381-5383 (1992). Another is encoding with peptides, in which for example a peptide tag is sequentially constructed on the same bead as the candidate (which may also be a peptide), each unit of the tag being a tripeptide sequence unique to the amino acid it encodes. The tags are separately decoded by Edman sequencing. A description of this type of encoding is presented by Kerr, J. M., et al., “Encoded combinatorial peptide libraries containing nonnatural amino acids,” Proc. Natl. Acad. Sci. USA 115: 2529-2531 (1993). Still another is the use of “hard” tags (chemically stable tagging moieties that are less labile than DNA and peptide tags) consisting of haloaromatic reagents linked to the beads through photochemically cleavable linkers. Once liberated, the tags can be detected using electron capture capillary gas chromatography. Other hard tags may involve amino acids in binary encoding in which unique information is obtained from either the presence or absence of a given amino acid. A description of this type of tagging technology is presented by Ohlmeyer, M. H. J., et al., “Complex synthetic chemical libraries indexed with molecular tags,” Proc. Natl. Acad. Sci. USA 90: 10922-10926 (1993). Radio-frequency encoding technologies can also be used, using rf transponders implanted in the beads, and scanning the beads after the assay to read the unique rf signature of a given bead. The signature may for example consist of a two-dimensional array of dots machined into the bead surface by laser drilling techniques, each dot representing a binary signal by its presence or absence in a specific location in the array, the orientation of the array and the registration of the image controlled by edge or corner fiducials in the array. Descriptions of this type of technology are found in Moran, E. J., et al., “Radio-frequency tag encoded combinatorial library method for the discovery of tripeptide-substituted cinnamic acid inhibitors of the protein tyrosine phosphatase PTP1B,” J. Am. Chem. Soc. 117: 10787-10788 (1995); Mandecki, W., U.S. Pat. No. 5,641,634, “Electronically-indexed solid-phase assay for biomolecules,” issued Jun. 24, 1997; and Mandecki, W., (Pharmaseq, Inc.) U.S. Pat. No. 5,981,166, “Screening of soluble chemical compounds for their pharmacological properties using transponders,” issued Nov. 9, 1999, and the various references cited in these documents. All of the citations in this paragraph are incorporated herein by reference.

The use of encoding technologies in the practice of the present invention may be understood by a description of a single technology as an example. According to this technology, identifier tags are used that consist of sequences of monomeric units differing from those of the constructs used as test candidate species, each monomeric unit of a tag corresponding to the monomeric unit of the construct that occupies the corresponding position in the sequence. The tags are formed in sequence on the microbeads simultaneously with the candidate constructs but at different regions on the microbead surface. The monomeric units of the tags may be joined together by different chemistries than those used to form the candidate constructs, to ensure that each monomeric unit of the tag becomes linked only to the tag and not to the candidate construct, and that each monomeric unit of the candidate construct links only to the construct and not the tag. As an illustration, for candidate constructs that are peptides in which each monomeric unit is an amino acid, the tags may consist of codons or three-nucleic acid sequences that correspond to the amino acids of the construct according to the genetic code, with different bonding chemistries for the peptide linkages and the nucleotide linkages. Other mutually exclusive chemistries can be used for other construct/tag combinations, and the various possibilities will be readily apparent to those skilled in the art.

The tags of the preceding paragraph are linked to the microbead surface by linkages that differ from those joining the candidate constructs so that the constructs can be released from the microbeads first, leaving the tags still linked to the microbeads. The tags can then be released from the microbeads at will, when it is desired to read a particular tag and thereby determine the sequence of monomeric units that constitute the particular construct to which the tag is associated. In addition, the linkages joining the constructs to the microbead surfaces are selected such that the constructs can be released from the microbeads without cleaving the linkages between units in the construct, i.e., while leaving the construct sequence intact. Likewise, the linkages joining the tags to the microbead surfaces are selected such that the tags can be released from the microbeads without cleaving the linkages between units in the tags, i.e., while leaving the tag sequence intact. Using microbeads and tags of this description, the candidate constructs can be released from the microbeads for use at the stage in the assays where the candidate constructs are to be placed in contact with the biological cells for possible mediation of the cellular response. Once an active construct is identified, the corresponding bead is readily recovered and the tag released for analysis.

Further descriptions of the various materials, chemistries, and methods for the use of microbeads and tags as described in the preceding paragraphs can be found in the following documents:

co-pending U.S. patent application Ser. No. 09/028,126, filed Feb. 23, 1998, entitled “Synthesizing and Screening Molecular Diversity,” William J. Dower et al., inventors

U.S. Pat. No. 5,639,603, issued Jun. 17, 1997, entitled “Synthesizing and Screening Molecular Diversity,” William J. Dower et al., inventors

U.S. Pat. No. 5,708,153, issued Jan. 13, 1998, entitled “Method of Synthesizing Diverse Collections of Tagged Compounds,” William J. Dower et al., inventors

U.S. Pat. No. 5,770,358, issued Jun. 23, 1998, entitled “Tagged Synthetic Oligomer Libraries,” William J. Dower et al., inventors

U.S. Pat. No. 5,789,162, issued Aug. 4, 1998, entitled “Methods of Synthesizing Diverse Collections of Oligomers,” William J. Dower et al., inventors

International Patent Application No. WO 93/06121, published Apr. 1, 1993, entitled “Methods of Synthesizing Diverse Collections of Oligomers,” Affymax Technologies N.V., applicant

International Patent Application No. WO 95/12608, published May 1, 1995, entitled “Synthesizing and Screening Molecular Diversity,” Affymax Technologies N.V., applicant

The entire disclosures of each of these documents are incorporated herein by reference for all legal purposes capable of being served thereby.

Once the microbeads with candidate constructs and identifier tags are prepared by the methods referenced above, the microbeads can be encapsulated with target species (either molecular species or biological cells) and intelligent polymers by methods known in the art. According to one such method, the microbeads and target species are suspended in liquid media containing the polymer precursors. The suspension is then forced through a pulsation chamber either by a syringe pump or by air pressure while vibration is applied to the chamber. The vibration is produced by a magnet and an electrical coil extending over the top of the chamber and arranged such that when alternating current is passed through the coil, the coil produces electromagnetic waves that interact with the magnet causing the magnet to vibrate, the vibrations being transmitted to the suspension. The vibrated suspension passes through a nozzle that separates the suspension into droplets of equal size, the microbead concentration and the droplet size being selected such that each droplet contains one microbead. The droplets emerging from the nozzle pass through an electric field between the nozzle and an electrode, the electric field producing a surface charge on the droplets. The charge creates electrostatic repulsion between the droplets which then fall into a hardening solution which causes the droplets to polymerize into capsules, the repulsive charge preventing the droplets and hence the capsules from aggregatiing. As an example of the typical parameters of such a system, a nozzle can be used that has a diameter ranging from about 80 μm to about 1,000 μm, with a voltage ranging from about 400 V to about 1700 V. The nozzle diameter has the strongest influence on the capsule size, while a degree of variability is introduced by the jet velocity and the vibration frequency. In general, the capsule diameter is approximately twice the nozzle diameter. Preferred conditions will produce capsules having a diameter ranging from about 160 μm to about 2,000 μm at a rate of from about 300 to about 4,000 per second.

Apparatus in which capsules can be formed in this manner can be obtained from various commercial suppliers, examples of which are Inotech Biosystems International, Inc., of Rockville, Md., USA, and Inotech AG, of Dottikon, Switzerland. Descriptions of the apparatus and its methods of use are found in International Patent Application No. WO 99/44735, entitled “Method and Device for Encapsulating Microbial, Plant and Animal Cells or Biological and Chemical Substances,” Inotech AG, applicant, published Sep. 10, 1999. The contents of WO 99/44735, are incorporated herein by reference.

By application of the methods described above, the present invention can be used for high-throughput screening, i.e., the screening of large numbers of candidates (commonly referred to as “libraries”) in rapid and parallel manner, using automated equipment with highly controlled and uniform conditions and producing highly sensitive, reliable and accurate results. Batches of compounds may for example be tested simultaneously for binding activity or biological activity against target molecules, i.e., as inhibitors of target enzymes, as competitors with a natural ligand for binding to the receptor of the ligand, as agonists or antagonists for receptor-mediated intracellular processes, and various other cellular and biological functions. High-throughput screening methods in accordance with this invention may serve as complete screening methods by themselves, or as the first stage of multistage screening procedures. Positive high throughput screening results, commonly referred to as “hits,” may thus identify candidates for further testing stages. For example, enzyme inhibitors identified through hits in a first screening stage may be screened in a second stage to select those of a particular potency, or compounds active as ligands for a particular receptor can be identified in a first stage and then screened in a second stage to identify those of a particular binding affinity. A candidate that succeeds in the first or second stage may be used as a lead compound for directing the synthesis of structurally related compounds for further screening to determine if even more successful candidates can be identified. On the other hand, candidates that fail one or more the screening stages can be returned to the library and saved for screening against other targets. The methods of this invention thus find use in a wide variety of screening protocols.

The foregoing is offered primarily for purposes of illustration. Further variations, modifications, and alternatives of the materials, components, operating conditions, and procedural steps that are still within the spirit and scope of the invention will be apparent to those skilled in the art.

What is claimed is:

1. A method of screening a candidate species for its ability to evoke a response in a target that produces a change in the environment of said target species, said method comprising:
(a) forming a capsule containing said candidate species with said target and an intelligent substance which is defined as a substance that undergoes a transformation upon exposure to said change in environment, said candidate species and said target isolated from each other in said capsule and yet capable of being placed in contact by an externally imposed condition;
(b) imposing said condition on said capsule, thereby placing said candidate species and said target in contact; and
(c) monitoring said capsule for an indication of said transformation in said intelligent substance.

2. A method in accordance with claim 1 in which said target is a biological cell.

3. A method in accordance with claim 1 in which said target is an enzyme.

4. A method in accordance with claim 1 in which said target is a biological receptor.

5. A method in accordance with claim 4 in which said biological receptor is an intracellular receptor selected from the group consisting of estrogen receptors, glucocorticoid receptors, androgen receptors, progesterone receptors, and mineralocorticoid receptors.

6. A method in accordance with claim 1 in which said target is a transcription factor.

7. A method in accordance with claim 1 in which said target is a kinase.

8. A method in accordance with claim 1 in which said target is a member selected from the group consisting of proteins, sugars, nucleic acids, and lipids.

9. A method in accordance with claim 1 in which said target is an enzyme and said response is inhibition of said enzyme.

10. A method in accordance with claim 1 in which said target is a biological receptor and said response is activation of said receptor.

11. A method in accordance with claim 1 in which said target is a biological receptor and said response is an inhibition of the activation of said receptor due to competition for said receptor between said candidate species and a natural activator of said receptor.

12. A method in accordance with claim 1 in which said target is a biological cell and said response is agonist action on a receptor of said biological cell and a consequent intracellular process mediated by said agonist action.

13. A method in accordance with claim 1 in which said target is a biological cell and said response is antagonist action on a receptor of said biological cell and a consequent diminishment of an intracellular process due to said antagonist action.

14. A method in accordance with claim 1 in which said candidate species is a single molecular species.

15. A method in accordance with claim 1 in which said candidate species is a combination of molecular species.

16. A method in accordance with claim 1 in which said indication of said transformation in said intelligent substance is a member selected from the group consisting of a change in size of said capsule, a change in shape of said capsule, a change in an optical property of said intelligent substance, a release of a detectable species from said capsule, and an emission of a detectable signal from said capsule.

17. A method in accordance with claim 16 in which said indication of said transformation in said intelligent substance is a change in size of said capsule.

18. A method in accordance with claim 16 in which said indication of said transformation in said intelligent substance is a change in shape of said capsule.

19. A method in accordance with claim 1 in which said intelligent substance is a crosslinked polymer and said change in environment is the release of an agent that disrupts the crosslinking in said polymer, thereby causing said polymer to swell.

20. A method in accordance with claim 19 in which said polymer is crosslinked by antigen-antibody interaction between polymer chains to which antigen and antibody are covalently bound, and said change in environment is the release of free antigen or antibody that competes with said antigen-antibody interaction between polymer chains.

21. A method in accordance with claim 19 in which said polymer is crosslinked by a linking group that is cleavable by an enzyme, and said change in environment is the release of said enzyme.

22. A method in accordance with claim 21 in which said linking group is a β-galactoside, and said change in environment is the release of β-galactosidase.

23. A method in accordance with claim 16 in which said indication of said transformation in said intelligent substance is a change in fluorescence of said capsule.

24. A method in accordance with claim 23 in which said change in fluorescence of said capsule is a fluorescence emission from an otherwise non-fluorescing capsule or an increase in fluorescence emission from said capsule.

25. A method in accordance with claim 24 in which said intelligent substance comprises a support matrix with a fluorophore bound thereto, and said change in environment is the release of a cleaving agent that cleaves said fluorophore said support matrix, thereby causing a fluorescence emission.

26. A method in accordance with claim 25 in which said fluorophore is a 5-alkanoylaminofluorescein di-β-galactopyranoside, and said cleaving agent is β-galactosidase.

27. A method in accordance with claim 24 in which said intelligent substance comprises a crosslinked polymer with a fluorescence resonance energy transfer pair bound thereto and spaced apart to produce no net fluorescence, and said change in environment is the release of an agent that disrupts the crosslinking in said polymer, thereby changing the spacing of said energy transfer pair and producing a net fluorescence.

28. A method in accordance with claim 1 in which said candidate species is a chemical compound.

29. A method in accordance with claim 28 in which said candidate compound is releasably immobilized on a solid support and said target is shielded from said solid support by a barrier that is impermeable to said solid support yet permeable to said candidate compound, and said externally imposed condition is a condition that causes release of said candidate compound from said solid support.

30. A method in accordance with claim 29 in which said solid support is a bead whose longest linear dimension is from about 1 nm to about 1 mm.

31. A method in accordance with claim 29 in which said solid support is a bead whose longest linear dimension is from about 0.5 μm to about 500 μm.

32. A method in accordance with claim 29 in which said candidate compound is covalently bonded to said solid support, and said externally imposed condition is a condition that causes cleavage of said candidate compound from said solid support.

33. A method in accordance with claim 32 in which said candidate compound is covalently bonded to said solid support through a nucleic acid linking group with a restriction site and step (b) comprises impregnating said capsule with a restriction enzyme effective to cause cleavage at said restriction site.

34. A method in accordance with claim 32 in which said candidate compound is covalently bonded to said solid support through a photocleavable linking group and step (b) comprises irradiating said capsule with light at a wavelength effective to cause cleavage of said linking group.

35. A method in accordance with claim 34 in which said photocleavable linking group is cleavable by ultraviolet light, said barrier shielding said biological cell from said solid support is a casing that is impermeable to ultraviolet light, and step (b) comprises irradiating said capsule with ultraviolet light.

36. A method in accordance with claim 1 in which step (a) comprises encasing said candidate species in a shell and forming said capsule around said shell with said target species external to said shell, and said externally imposed condition is a condition that renders said shell permeable to said candidate species.

37. A method in accordance with claim 36 in which said shell is absorptive of electromagnetic radiation and rupturable by heat and step (b) comprises irradiating said shell with electromagnetic radiation.

38. A method in accordance with claim 36 in which said shell encasing said candidate species is impermeable to said candidate species and is defined as a first shell and said target is surrounded by a second shell which is permeable to said candidate species and outside said first shell, said first shell being light absorptive and rupturable by heat and said second shell being transparent, and step (b) comprises irradiating said capsule with light thereby causing said first shell to rupture due to heat caused by light absorption without rupture of said second shell.

39. A method in accordance with claim 36 in which said shell encasing said candidate species is impermeable to said candidate species and said target is embedded in a matrix that is outside said shell and permeable to said candidate species, and step (b) comprises rupturing said shell.

40. A method in accordance with claim 36 further comprising encasing a plurality of magnetic particles in said shell with said candidate species, and step (b) comprises imposing a magnetic field on said capsule causing said magnetic particles to align and thereby rupture said shell.

41. A method in accordance with claim 36 further comprising encasing a plurality of magnetic particles in said shell with said candidate species, and step (b) comprises imposing a magnetic field on said capsule to impart oscillating movement to said magnetic particles, said movement causing rupture of said shell.

42. A method in accordance with claim 36 in which said shell comprises a material that is contractible upon exposure to an external stimulus, and step (b) comprises exposing said shell to said external stimulus to cause contraction of said material sufficient to form channels through said shell for escape of said candidate species.

43. A method in accordance with claim 36 further comprising encasing within said shell a solid particle which either contracts or expands upon exposing said shell to an external stimulus, and step (b) comprises exposing said shell to said external stimulus to cause sufficient contraction or expansion to rupture said shell.

44. A method in accordance with claim 1 in which step (a) comprises sequestering said candidate species in a swollen hydrogen and forming said capsule around said hydrogel with said target external to said hydrogel, and said externally imposed condition is a condition that causes said hydrogel to contract and thereby expel said candidate species for contact with said target.

45. A method of screening a plurality of candidate species for their ability to evoke a response in a target that produces a change in the environment of said target species, said method comprising:
(a) forming a plurality of capsules, each capsule containing one of said candidate species, a plurality of said target species, and an intelligent substance which is defined as a substance that undergoes a transformation upon exposure to said change in environment, said candidate species in each capsule isolated from said target in the same capsule and yet capable of being placed in contact with said target by an externally imposed condition;
(b) imposing said condition on said plurality of capsules, thereby placing said candidate species in contact with said target; and
(c) monitoring said capsules for an indication of said transformation in said polymer and identifying the candidate species contained in a capsule that exhibits said indication.

46. A method in accordance with claim 45 in which each candidate species is a single molecular species.

47. A method in accordance with claim 45 in which each candidate species is a combination of at least two distinct molecular species.

48. A method in accordance with claim 45 in which said candidate species are chemical compounds that are releasably immobilized on microbeads with each candidate compound on a separate microbead and at most one microbead retained in each capsule, and said externally imposed condition is a condition that causes release of candidate compounds from all of said microbeads.

49. A method in accordance with claim 45 in which said candidate species are chemical compounds that are releasably immobilized on microbeads on which are also immobilized identifier tags, each microbead having immobilized thereon a single candidate compound and a single identifier tag which comprises a detectable code that is decipherable to indicate the molecular structure of the candidate compound, and said externally imposed condition is a condition that causes release of all of said candidate compounds from said microbeads.

50. A method in accordance with claim 45 in which said candidate species are peptides.

51. A method in accordance with claim 49 in which said candidate species are peptides and each identifier tag is an oligonucleotide comprised of a sequence of codons corresponding to the sequence of amino acids in the peptide immobilized on the same microbead as said tag.

52. A method in accordance with claim 49 in which said candidate species are chemical compounds and are immobilized on said microbeads in a manner that permits release of said chemical compounds without releasing said identifier tags.
